(19) United States
(12) Patent Application Publication
(10) Pub. No.: US 2004/0247639 A1
(43) Pub. Date: Dec. 9, 2004

(54) Novel methods and compositions for the treatment or prevention of dysmenorrhoea and menstrual side effects: the use of phospholipase inhibitors

(76) Inventors: Ian Alexander Shiels, Queensland (AU); Stephen Maxwell Taylor, Queensland (AU); David Paul Fairlie, Queensland (AU)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/483,898

(22) PCT Filed: Jul. 13, 2001
(86) PCT No.: PCT/AU01/00858

(30) Foreign Application Priority Data
Jul. 14, 2000 (AU) PQ8764

(51) Int. Cl. A61K 31/66; A61K 9/70

(52) U.S. Cl. 424/423; 514/114

(57) ABSTRACT

The present invention discloses the use of phospholipases A₂ inhibitors in compositions and in methods for the treatment and/or prophylaxis of dysmenorrhoea, menstrual migraine and menorrhagia.

### FIELD OF THE INVENTION

THIS INVENTION relates generally to compositions and methods for modulation of uterine contractions and for reducing or alleviating discomforting symptoms such as pain and blood loss. More particularly, the present invention relates to compositions comprising a molecule which inhibits a phospholipase and in particular a phospholipase A₂ enzyme and more particularly a secretory phospholipase A₂ from an animal or mammal and to methods of using such compositions for the treatment and/or prophylaxis of dysmenorrhoea and related conditions. The invention further relates to use of the compositions of the invention for the treatment and/or prophylaxis of premature uterine expulsion of a foetus or embryo, impending abortion or miscarriage.

Biographical details of various publications referred to in this specification are collected at the end of the description.

### BACKGROUND OF THE INVENTION

Fifty percent of menstruating women suffer from dysmenorrhoea, the painful “period pain” accompanying menstruation, and this represents a substantial social and economic problem for the community. In the United States alone it has been estimated that 600 million work hours are lost to dysmenorrhoea each year. (Dawood, M Y, 1988, Am J Med, 84: 23-29). In Britain it has been estimated that 3.8 million women suffer dysmenorrhoea every period, a further 1.6 million regularly suffer dysmenorrhoea during their periods and 1.4 million suffer occasionally. Although the pathology of primary dysmenorrhoea has an uncertain cause, it has been shown that the clinical signs of dysmenorrhoea do correlate with plasma and menstrual fluid levels of inflammatory mediators (termed eicosanoids) that are synthesised in the uterus (Prigent et al, 1994, Prostaglandins, 47: 451-66). Eicosanoids such as prostaglandins (PGs) and leukotrienes (LTs) are normally the physiological mediators of uterine contraction (Lopez Bernal et al, 1989, Br J Obstet Gynaecol, 96: 568-73), but in some women these chemicals are over-produced, their concentrations exceed physiological requirements, and clinical signs of dysmenorrhoea begin to develop (Benedetto, 1989, Gynecol Endocrinol, 3: 71-94)

As part of normal physiological function, the uterus contracts rhythmically and the force and frequency of these contractions are regulated, in the first instance, by sex hormones (Prigent et al, 1994, Prostaglandins, 451-66). Changes in function accompany changes in uterine structure throughout the sexual (menstrual) cycle to prepare the uterus for implantation of a conceptus. If pregnancy does not occur the uterine lining is shed at menstruation in preparation for another reproductive cycle. Sex hormones can regulate uterine contractility by regulating specialised cells in the uterine wall which then synthesise eicosanoids that act directly on uterine muscle cells (myometrium) to cause contraction (Prigent et al, 1994, Prostaglandins, 451-66).

Primary dysmenorrhoea is an abnormality resulting in the secretion of greater than normal levels of eicosanoids in the uterus. Elevated concentrations of all classes of eicosanoids (PGs, LTs, hydroxyeicosatetraenoic acids (HETEs)), as well as platelet activating factor (PAF), have been identified in women suffering dysmenorrhoea (Nigam,S et al, 1991, Eicosanoids; 4: 137-141, Zahradnik,H P & Brechwood, M, 1984, Arch Gynecol 236: 99-108, Bieglmayer, C et al, 1995, Gynecol Endocrinol, 9: 307-312). Such elevated concentrations of these mediators increase the force of uterine contraction (cramping), constriction of blood vessels with resultant anoxia of tissues (pain), and the sensitisation of pain receptors in pelvic nerve terminals to other pain-inducing chemicals and physical stimuli (Benedetto, C, 1989, Gynecol Endocrinol, 3: 71-94). The eicosanoids can enter the circulation and cause diarrhoea, headache, dizziness, nausea and inflammation.

Current treatment for dysmenorrhoea relies mainly on the use of aspirin or non-steroidal anti-inflammatory drugs (NSAIDs). In approximately 80% of women with dysmenorrhoea the symptoms are relieved to some extent by treatment with a non-steroidal anti-inflammatory drug (NSAID) which inhibits the cyclooxygenase enzymes (COX-1 or COX-2) that synthesise prostaglandins from arachidonic acid (FIG. 1). Prolonged use of NSAIDs does however cause serious gastrointestinal side effects in humans (Whittle, B J, 1979, Acta Ostet Gynecol Scand Suppl, 87: 21-26) and in at least 20% of primary dysmenorrhoea patients, NSAIDs do not give satisfactory relief (Dawood, M Y, 1988, Am J Med, 84: 23-29, Whittle, B J, 1979, Acta Ostet Gynecol Scand Suppl, 87: 21-26).

The contraceptive pill, which regulates the levels of the natural sex hormones to suppress ovulation, is also used to treat dysmenorrhoea in women who wish contraception. However, there is currently no effective drug therapy for women who cannot take either the contraceptive pill or who get no relief from NSAIDs. In this respect, some women are unable to take NSAIDs eg, aspirin sensitive asthmatics, and some women are unable to tolerate the contraceptive pill.

Two other side effects of menstruation are menstrual migraine and menorrhagia. Menstrual migraine is a headache that is triggered during the menstrual cycle and, like dysmenorrhoea, has been shown to be caused by changes in levels of prostaglandins (Nattero, G, et al, 1989, Headache, 29: 233-238). Menorrhagia is excessive bleeding occurring in 9-14% of healthy women and may be associated with dysmenorrhoea (van Eijkeren, MA et al, 1992, Drugs, 43: 201-209). This condition may be treated with hormones either orally or by medicated intrauterine device (IUD), antifibrinolytics and NSAIDs (Bonner,J and B L Sheppard, 1996, BMJ, 313: 579-582, van Eijkeren, MA et al, 1992, Drugs, 43: 201-209).

Secondary dysmenorrhoea has a well-defined and identifiable pathology. Eicosanoids have been identified in the pathology of endometriosis (a cause of secondary dysmenorrhoea) where abnormal endometrial tissue produces elevated levels of prostaglandins in the peritoneum (Benedetto, C, 1989, Gynecol Endocrinol, 3: 71-94). Treatment of secondary dysmenorrhoea usually is aimed at modifying the primary pathology although drugs that block synthesis of autacoids (like NSAIDs) give symptomatic relief. NSAIDs are commonly used to ameliorate the signs of primary and some cases of secondary dysmenorrhoea as well as menorrhagia.

While a limited number of therapies are presently available for reducing or alleviating discomforting symptoms associated with dysmenorrhoea and associated menstrual side effects, there remains a need for more effective compositions and treatment methods for this purpose.

### SUMMARY OF THE INVENTION

The present invention arises in part from the unexpected discovery that inhibitors of phospholipases, particularly phospholipases A₂, and more particularly secretory phospholipases A₂, are up to 1000 times more effective in inhibiting uterine contractions compared to NSAIDs. The inventors consider that these inhibitors specifically inhibit the formation of mediators, including PGs, LTs, HETES and PAF, that cause uterine contraction. Not wishing to be bound by any one particular theory or mode of action, it is believed that these mediators, either alone or in combination, may promote disorders associated with the menstrual cycle, including dysmenorrhoea, menstrual migraine and menorrhagia. The foregoing discovery has been reduced to practice in novel compositions and methods for reducing or alleviating the discomforting symptoms associated with menses and more particularly with dysmenorrhoea and associated menstrual side effects and for the treatment and/or prophylaxis of other conditions as described hereinafter.

Accordingly, in one aspect of the present invention there is provided a mense-related symptom-alleviating composition comprising a phospholipase inhibitor, together with a pharmaceutically acceptable carrier.

Suitably, the phospholipase inhibitor is a secretory phospholipase A₂ inhibitor. In a preferred embodiment of this type, the secretory phospholipase A₂ (sPLA₂) inhibitor is a non-pancreatic sPLA₂ inhibitor and more preferably a human non-pancreatic sPLA₂ inhibitor. In an especially preferred embodiment, the non-pancreatic sPLA₂ inhibitor is an sPLA₂ type IIa inhibitor.

The mense-related symptom includes, but is not restricted to, uterine contractions, pain and blood loss. Preferably, the mense-related symptom is associated with dysmenorrhoea, menstrual migraine or menorrhagia.

In a preferred embodiment, the composition is formulated for topical or local administration. In another preferred embodiment, the composition is formulated for oral administration.

In another aspect, the invention resides in a method for reducing or alleviating a mense-related symptom, comprising administering to a patient in need of such treatment an effective amount of a composition as broadly described above.

In another aspect, the invention provides a method for the treatment and/or prophylaxis of a condition selected from the group consisting of dysmenorrhoea, menstrual migraine and menorrhagia, said method comprising administering to a patient in need of such treatment or prophylaxis an effective amount of a composition as broadly described above.

Preferably, the composition is administered prior to the onset of menstruation.

According to another aspect of the invention there is provided a tocolytic composition comprising a phospholipase inhibitor as broadly described above, together with a pharmaceutically acceptable carrier.

In yet another aspect, the invention contemplates a method of modulating uterine contractions in a patient, comprising administering to a patient in need of such modulation an effective amount of a tocolytic composition as broadly described above.

In yet another aspect of the invention there is provided a method for modulating uterine expulsion of an embryo, comprising administering to a patient in need of such modulation an effective amount of a tocolytic composition as broadly described above.

The invention also encompasses the use of a phospholipase inhibitor as broadly described above in the preparation of a medicament for treating or preventing a condition selected from the group consisting of dysmenorrhoea, menstrual migraine, menorrhagia, premature uterine expulsion of a foetus or embryo, impending abortion and miscarriage.

In a related aspect, the invention contemplates the use of a phospholipase inhibitor as broadly described above in the preparation of a medicament for treating or preventing a condition selected from the group consisting of dysmenorrhoea, menstrual migraine and menorrhagia.

In yet another aspect, the invention envisions the use of an agent that modulates the synthesis of arachidonic acid and/or lyso-platelet-activating factor in the preparation of a medicament for treating a mense related symptom, for modulating uterine contractions or for treating or preventing a condition selected from the group consisting of dysmenorrhoea, menstrual migraine, menorrhagia, premature uterine expulsion of a foetus or embryo, impending abortion and miscarriage.

### BRIEF DESCRIPTION OF THE DRAWINGS

FIG. 1 is a schematic representation of the inflammation cascade showing phospholipase A₂—mediated formation of eicosanoids such as arachidonic acid, platelet activating factor (PAF), prostaglandins (PG), leukotrienes (LT), lipoxins (LX), thromboxanes (Tx), and lysophosphatides.

FIG. 2 is a schematic representation showing some known inhibitors of human non-pancreatic secretory PLA₂ (type IIa).

FIG. 3 shows Dixon plots for the determination of potencies of compounds 5 and 9 as inhibitors of human non-pancreatic sPLA₂ (type IIa).

FIG. 4 is a graphical representation showing the effects of human non-pancreatic sPLA₂ inhibitor 5 on the spontaneous activity of rat uteri in the organ bath (n=3, EC₅₀ 30 nM).

FIG. 5 is a graphical representation showing the effects sPLA₂ inhibitor 5 on the uterine contractions induced by oxytocin (n=3, ED₅₀ 20 nM).

FIG. 6 is a graphical representation showing the effects sPLA₂ inhibitor 9 on the spontaneous contraction and oxytocin induced contractions in rat uterus (n=2, EC₅₀ approx. 100 nM).

FIG. 7 is a graphical representation showing the effects of flunixin meglumine on spontaneous rat uterine contractions and oxytocin-induced contractions. Flunixin reduced spontaneous contractions at 1 μM and 5 μM concentrations, respectively, but had no effect on oxytocin-induced contractions (n=3, *P>0.05, **P.0.01).

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “alleviating” as used herein refers to prophylactically treating a woman from incurring a mense-related symptom, holding in check such symptoms, and/or treating existing symptoms.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

By “pharmaceutically-acceptable carrier” is meant a solid or liquid filler, diluent or encapsulating substance that may be safely used in topical, local or systemic administration.

By “effective amount”, in the context of treating or preventing a condition is meant the administration of that amount of active to an individual in need of such treatment or prophylaxis, either in a single dose or as part of a series, that is effective for treatment of, or prophylaxis against, that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “prodrug” is used in its broadest sense and encompasses those compounds that are converted in vivo to a phospholipase A₂ inhibitor according to the invention. Such compounds would readily occur to those of skill in the art, and include, for example, compounds where a free hydroxy group is converted into an ester derivative.

2. Compositions of the Invention

The present invention is generally directed to compositions comprising a phospholipase inhibitor, and a pharmaceutically acceptable carrier. Surprisingly, these compositions have been found to inhibit uterine contractions and it is believed that such inhibition may also reduce or alleviate at least one mense-related symptom including pain and blood loss. In a preferred embodiment, the mense-related symptom is associated with dysmenorrhoea, menstrual migraine or menorrhagia. Thus, the invention also encompasses a method for reducing or alleviating a mense-related symptom, comprising administering to a patient in need of such treatment an effective amount of a composition as broadly described above. In a preferred embodiment, the composition is administered immediately prior to the onset of menstruation.

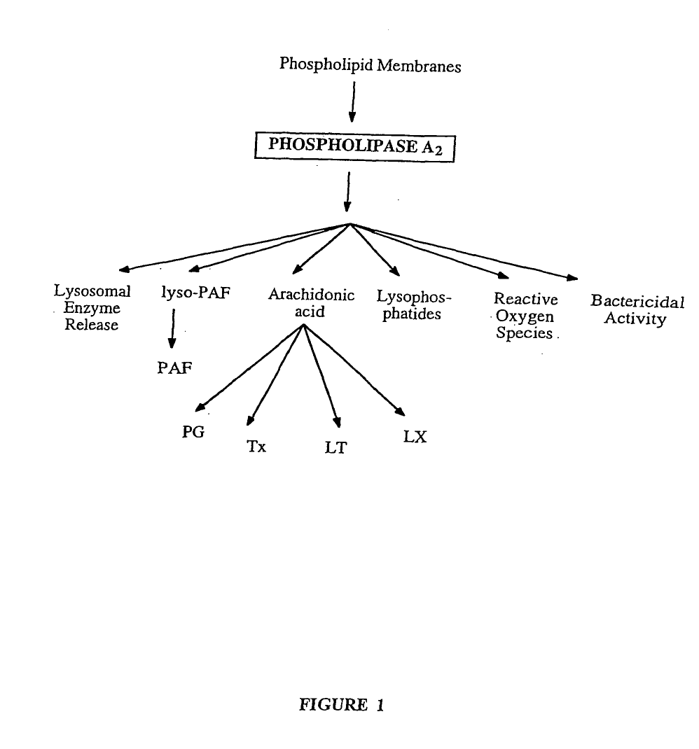

Not wishing to be bound by any one particular theory or mode of action, it is believed that the compositions of the present invention effectively modulate the synthesis of effectors of uterine contraction (ie, eicosanoids and PAF), by directly modulating the synthesis of arachidonic acid and lyso-platelet-activating factor from which these effectors derive (see FIG. 1).

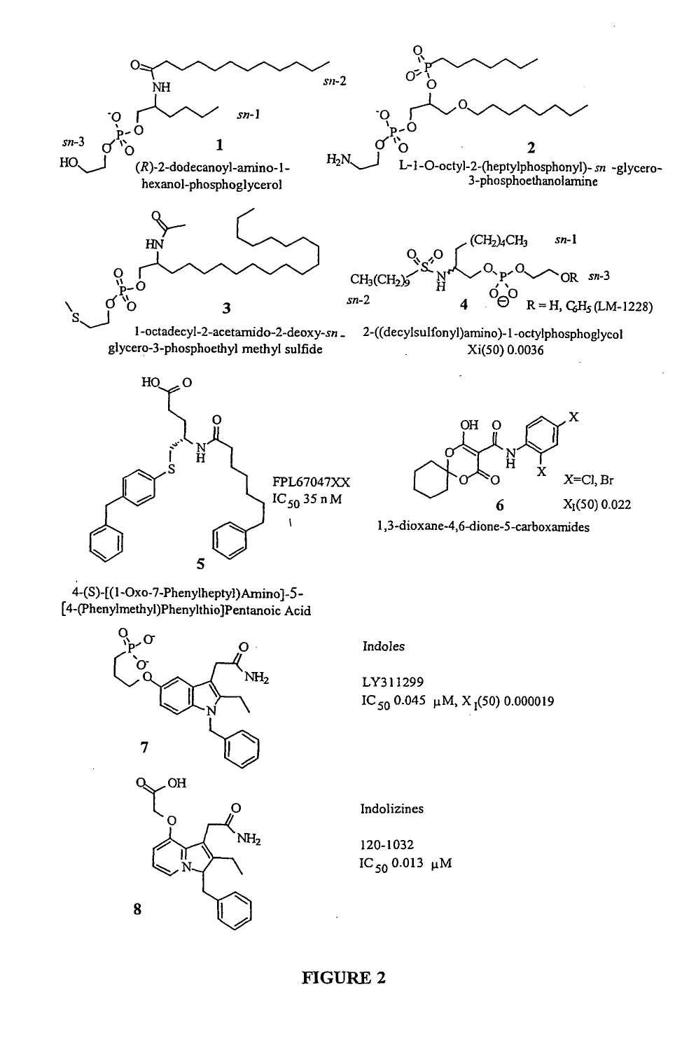

The present invention extends to any molecules which inhibit phospholipase and in particular a phospholipase A₂ enzyme and more particularly a secretory phospholipase A₂ from an animal or mammal. Animals and mammals contemplated by the present invention include humans, primates, livestock animals, companion animals, reptiles, insects and arachnids. Any suitable inhibitor of secretory phospholipases A₂ (sPLA₂) is contemplated by the present invention. In a preferred embodiment, the phospholipase inhibitor is a non-pancreatic sPLA₂ (e.g., type IIa) inhibitor. Examples of such inhibitors include compounds 1-8 presented in FIG. 2, or compound 9 described in Example 2, or derivatives, homologues, functional equivalents, pharmaceutically acceptable salts or prodrugs thereof. In an especially preferred embodiment, the sPLA₂ inhibitor is a human non-pancreatic sPLA₂ inhibitor. Currently there are four known and well-characterised types of human non-pancreatic sPLA₂ enzymes (Dennis, E. A., 1997, Trends Biochem. Sci., 22: 1-2; Balboa. M. A. et al, 1996, J. Biol. Chem. 271 (50): 32381-32384; Murakami, M. et al, 1999, J. Biol. Chem. 274 (44): 31435-31444; Hanasaki, K. et al, 1999, J. Biol. Chem. 274 (48): 34203-34211) but more are expected to be characterised soon. Type IIa is found in human platelets and synoviocytes. Type IId has been isolated from the spleen. Type V is found in human macrophages. Type X has been isolated from splenic leucocytes and thymocytes. It is anticipated that many other sPLA₂ enzymes remain to be discovered in other human cells and tissues. In a preferred embodiment, the sPLA₂ inhibitor is an sPLA₂ type IIa inhibitor.

Based on their efficacy to modulate uterine contractions, phospholipase inhibitors as broadly described above are also considered to be efficacious in tocolytic compositions for suppression of uterine contractions. The use of tocolytic drugs is well established in veterinary medicine for the management of birth in animals such as cattle. By controlling the time of birth through modulation of uterine contraction, assistance can be provided to ensure live young.

The compositions of the invention can also be used as a means of blocking premature uterine expulsion of an embryo or foetus that occurs during an impending abortion caused, for example, by abnormal hormone levels and infection of the placenta, or that may occur during in vitro fertilisation techniques. The compositions described herein may also be used as adjuncts in reducing uterine contractions associated with miscarriage.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the condition and preferably at least one mense-related symptom. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the condition, the physician may evaluate uterine contractions, uterine, vaginal or migraine pain, or blood loss. In any event, those of skill in the art may readily determine suitable dosages of the therapeutic agents of the invention.

Depending on the specific conditions being treated, the phospholipase inhibitor may be formulated and administered systemically, topically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Local or topical routes are preferred for administering the compositions of the invention. In this instance, the subject compositions may be formulated in any suitable manner, including, but not limited to, creams, gels, oils, ointments, solutions and vaginal suppositories. Alternatively, the said compositions may be applied in association with any suitable intravaginal or intracervical device, such as a tampon, diaphragm, sponge, or membrane (with or without a pessary).

Tampons used for selective expulsion or delivery of medicaments and other materials into the vaginal cavity are well known in the art, and may be used in the practice of this invention (see, e.g., U.S. Pat. Nos. 5,273,521, 4,309,997 and 4,318,405). Suitable tampons typically contain an absorbent material, and a composition of this invention may be inserted into the vaginal cavity followed by the tampon to prevent leakage. Alternatively, a composition of this invention may be impregnated onto or encapsulated within a tampon or other suitable applicator for delivery purposes. (See, e.g., U.S. Pat. No. 5,299,581).

Other devices, such as that disclosed in U.S. Pat. No. 5,299,581 may be used in conjunction with tampons and provide a means for administering a composition of this invention that might otherwise leak out. Reference also may be made to U.S. Pat. No. 5,527,534 in which a sterile, vaginal sponge delivery system is disclosed suitable for the sustained release of a composition of this invention.

The compositions of this invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

From the foregoing, the compositions of the invention may be administered locally to the uterus via intravaginal or intracervical application. In one embodiment, the composition may be administered in association with a tampon. Compositions administered in this manner will diffuse into the uterus. In another embodiment, the subject compositions may be applied to the surface of the lower abdomen, and diffuse through the skin to the uterus. Topical administration in this manner may be enhanced through the use of ultrasound or iontophoresis, or combination thereof Alternatively, the phospholipase inhibitors can be formulated readily using pharmaceutically acceptable carriers well known in the art into dosages suitable for oral administration, which is also preferred for the practice of the present invention. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or algiric acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, eg. by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical which can be used orally include push-fit capsules made of gelatine, as well as soft, sealed capsules made of gelatine and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic/prophylactic agents of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically/prophylactically effective dose can be estimated initially from cell culture assays. For example, a dose can be formulated in animal models to achieve a circulating concentration range that includes the IC50 as determined by 50% inhibition of the force and frequency of uterine contraction. Such information can be used to more accurately determine useful doses in humans.

Toxicity and therapeutic efficacy of such agents can be determined by standard pharmaceutical procedures in cell cultures or experimental animals, eg. for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent(s), which are sufficient to modulate uterine contractions, uterine, vaginal, or migraine pain, or blood loss. Usual patient dosages for systemic administration range from 1-4000 mg/day, commonly from 5-2500 mg/day, and typically from 50-500 mg/day. Stated in terms of patient body weight, usual dosages range from 0.02-75 mg/kg/day, commonly from 0.1-50 mg/kg/day, typically from 1-10 mg/kg/day.

In order that the invention may be readily understood and put into practical effect, particular preferred embodiments will now be described by way of the following non-limiting examples.

### EXAMPLES

### Example 1

Known sPLA₂ Inhibitors

There are several published X-ray crystal structures of human sPLA₂ enzyme (type IIa) complexed with an inhibitor. Examples of such inhibitors include compounds 1-7 presented in FIG. 2. Each of compounds 1-7 bind the enzyme within the same hydrophobic cavity defined by enzyme residues Cys27, Tyr22, Ala18, Ile9, His6, Phe5, Phe24, Gly23, Val31, Leu2, Tyr52 and Cys44. Each inhibitor is coordinated to the active site calcium ion via two oxygen atoms. Compound 2 is an example of a substrate analogue having a phosphonate in place of the cleavable ester in the substrate. It possesses the same components as the substrate but no cleavable bond, and the phosphorous atom is a “transition state analogue” since it mimics the tetrahedral carbon formed during ester hydrolysis. Inhibitors 1-4 (Thunnissen, 1990 MM., et al. 1990, Nature 347(6294): 689-91; Scott, D. L. et al, 1991, Science, 254: 1007-110; Oh,B.-H., 1995, Acta Cryst, D51: 140-144; Pisabarro, M. T. et al, 1994, J. Med. Chem., 37: 337-41) all bind in a similar manner with the sn-1 and sn-2 aliphatic chains occupying the hydrophobic channel and all make extensive hydrophobic interactions. The amide NH of 1 and 3 and the sulphonamide NH of 4 all form a hydrogen bond with the catalytic His48. The calcium binding phosphate group of the sn-3 chain of compounds 1-4 all form a hydrogen bond with Lys69 (Tyr, in the porcine PLA₂ complex with 1). The optimal chain length of the sn-1 alkyl chain is 4 carbons for binding to porcine pancreatic sPLA₂.(van den Berg L., et al, 1992 Biochim Biophlys Acta 1124(1): 66-70), while 10 carbons are preferred for the sn-2 chain for binding to cobra venom sPLA₂ (Yu, L. et al, 1992, J. Am. Chem. Soc., 114: 8757-8763). The human enzyme (Thunnissen, 1990, supra) prefers an aliphatic chain of 6 carbons as seen in inhibitor 5 (Cha, S. S., et al. 1996, J. Med. Chem., 39: 3878-81).

Inhibitor 5 has phenyl rings substituting for the common alkyl chains. It binds in the same way as 1-4 through calcium coordination and via a hydrogen bond to His48. The phenyl rings participate in extensive aromatic/aliphatic or aromatic/aromatic interactions with the non-polar amino acids lining the hydrophobic channel of the enzyme. They also interact intramolecularly with each other with the sn-2 phenyl ring aligning approximately perpendicularly to the two phenyl rings on the sn-1 chain and perpendicularly to His6. These aromatic or π-stacking interactions may carry significant importance in ligand binding. The carboxylate group in 5 was used to replace the phosphate group (1-4) to aid cell permeability demonstrated through a monocyte leukotriene release assay (IC₅₀ 0.35 μM). Several other phosphonate containing analogues showed no inhibitory activity at concentrations up to 50 μM.(Cha, 1996)

Inhibitor 6 is typical of carboxamides that bind directly to calcium via the enolate and the carboxamide carbonyl oxygens. The ortho substituent where X=Cl , makes polar van der Waals contact with the NH of His48 whilst the para chloro substituent is packed against the edges of the sidechains of Phe5 and Phe106 along with Gly23. The cyclohexyl ring makes interactions within the hydrophobic channel (Bryant, M.D. et al, 1999, Bioorg. Med. Chem. Lett., 9: 1097-102), Compounds 7 and 8 have indole and indolizine scaffolds connecting calcium-binding and hydrophobic inhibitor regions. Indoles like LY311727 (7) coordinate the calcium ion via the amide carbonyl oxygen at position 3 and a phosphonate oxygen at position 5. The phosphonate group also makes interactions with Lys69 via a water molecule. The amide NH at position 3 makes a characteristic hydrogen-bonding interaction with the catalytic His48. The phenyl heptanoyl group lies in the hydrophobic cavity making hydrophobic interactions with the enzyme and displaces His6 at the base of the cavity in a similar manner to that mentioned above. The indolizine compound, 8, binds like indole 7 to calcium via the amide carbonyl oxygen and via one oxygen from its carboxylate anion, which neutralises the positive charge on calcium. The amide NH is hydrogen bonded to the catalytic His48 as well as to the calcium-binding residue Asp49.(Kitadokoro, K. et al, 1998, J. Biochem, 123: 619-623)

### Example 2

Inhibitors of Human sPLA₂ are Inhibitors of Uterine Contractions

The present inventors have synthesised and tested compounds 5 and 9. Since many of the previously reported inhibitors of sPLA₂ are now known not to inhibit human non-pancreatic secretory PLA₂ (Balsinde J. et al, 1999, Annu Rev Pharmacol Toxicol, 39: 175-89), they first tested compounds 5 and 9 for their ability to inhibit human sPLA₂ (type IIa) before testing them as inhibitors of rat uterine contractions. Compound 5 has been reported previously (Cha et al, 1996, J. Med. Chem., 39: 3878-81) as an inhibitor of human non-pancreatic sPLA₂ (type IIa). Compound 9 is a derivative of compound 7, a known inhibitor (Schevitz, R. W. et al, 1995, Nature Structure Biology, 2(6): 458-465) of a human non-pancreatic sPLA₂ (type IIa). Compound 9 contains the same indole ring as 7 and was recently reported (Chen, Y. et al, 1998, Biochim. Biophys Acta, 1394: 57-64) to inhibit type V human non-pancreatic sPLA₂ (IC₅₀ 36 nM).

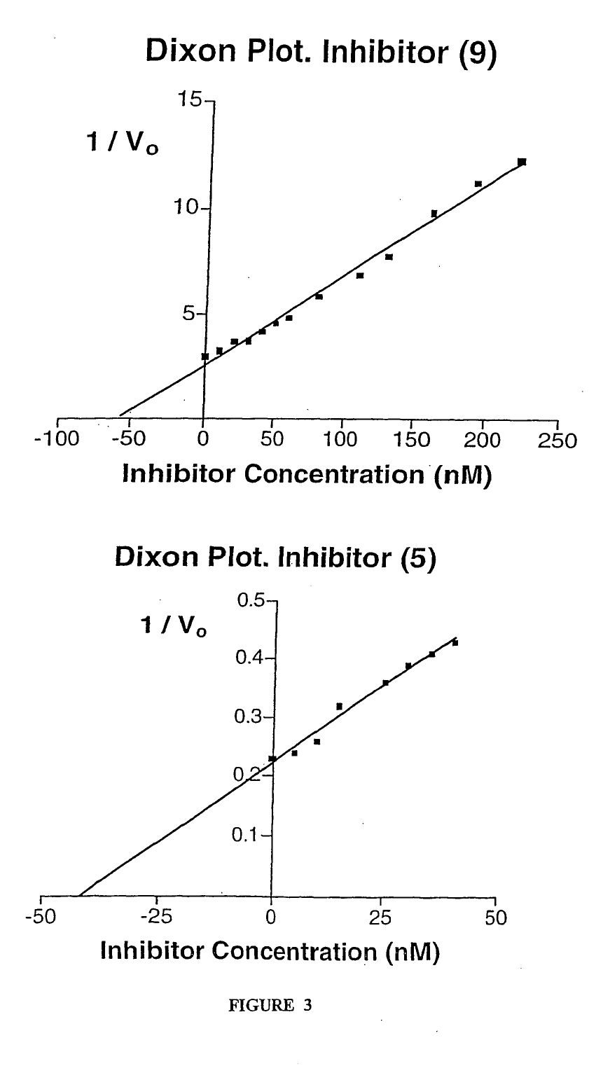

The inhibitor potencies of compounds 5 and 9 were measured against human non-pancreatic secretory PLA₂ using a rapid 96 well assay where each well has a total volume of 225 μL consisting of enzyme (110 ng sPLA₂), substrate (1.66 MM 1,2-Bis(heptanoylthio)-1,2-dideoxy-sn-glycero-3-phosphorylcholine (thio-PC), 0.3 mM Triton X-100 mixed micelles), buffer (25 mM Tris-HCL buffer (pH 7.5), 10 MM CaCl₂, 100 mM KCl, 1 mg/ml BSA) and DTNB (5-5′-dithiobis(2-nitrobenzoic acid). Mixtures were incubated with varying concentrations of test inhibitor at 37° C. and enzyme activity measured by a yellow colour due to thioester product (5-thio-2-nitrobenzoic acid), which is quantified by absorbance at 414 nm. Plots of A/t data allow measurements of initial reaction velocities, Kₘ, k_{cat}; IC₅₀ & Kᵢ values from Dixon plots. Under these conditions the inhibitor potencies of 5 and 9 were measured (FIG. 3) as IC50=42 nM and 60 nM respectively against the sPLA₂ enzyme. Thus both compounds 5 and 9 are confirmed as among the most potent known inhibitors of human secretory phospholipase A₂ (type IIa).

To examine the potential benefit of PLA₂ inhibitors in treating dysmenorrhoea in women, an in vitro model was developed which measured the force and frequency of contractions in a rat uterus. When removed from the rat and placed in an organ bath, the uterus contracts rhythmically. The force of contraction varies with the profile of sex hormones produced by the animal at the different stages of the sexual (oestrus) cycle. Levels of oestrogen and progesterone fluctuate throughout the sexual cycle. To ensure a reproducible model, rats were ovarectomised and oestrogen, progesterone, a combination of oestrogen and progesterone and an inert control substance were given to mimic different stages of the sexual cycle.

In this model the uterine contractions are stimulated by the synthesis and release of eicosanoids and other contractile mediators by cells in the uterus, which then act on the muscle cells in the uterine wall to cause contraction. Compounds 5 and 9 were tested for their ability to inhibit spontaneous and oxytocin-induced contractions of a female rat uterus (described ahead). Addition of oxytocin, a hormone synthesised in the pituitary gland, gives uterine contractions of greater force and frequency by increasing the release of eicosanoids (Wassdal, I et al, 1998, Acta Physiol Scand, 164: 47-52, Burns, P D et al, 1998, Domest Anim Endocrinol, 15: 477-487, Asselin, E et al, 1997, Endocrinology, 138: 4798-4805). There is a relationship between the stage of the sexual cycle and the sensitivity of the uterus to oxytocin (Engstrom, T et al., 1999, J. Endocrinol., 161: 403-411). It has been shown that the contractions can be reduced if the synthesis of the mediators is blocked.

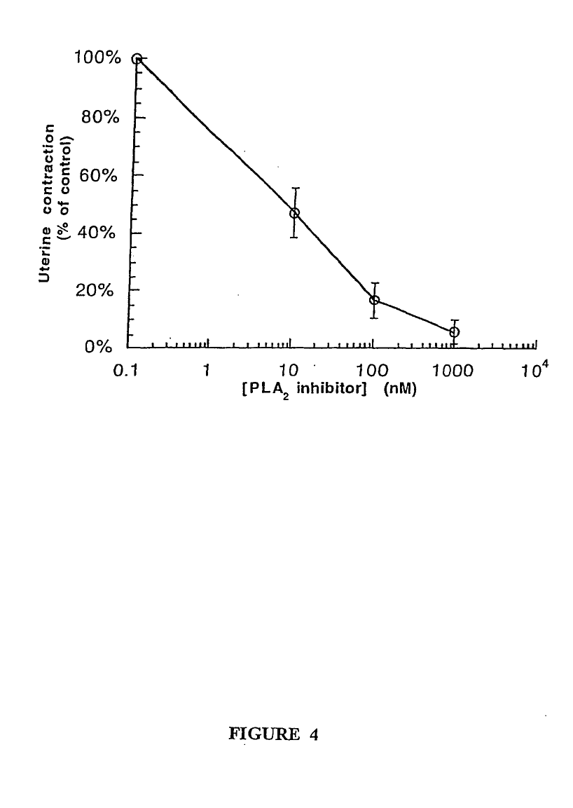

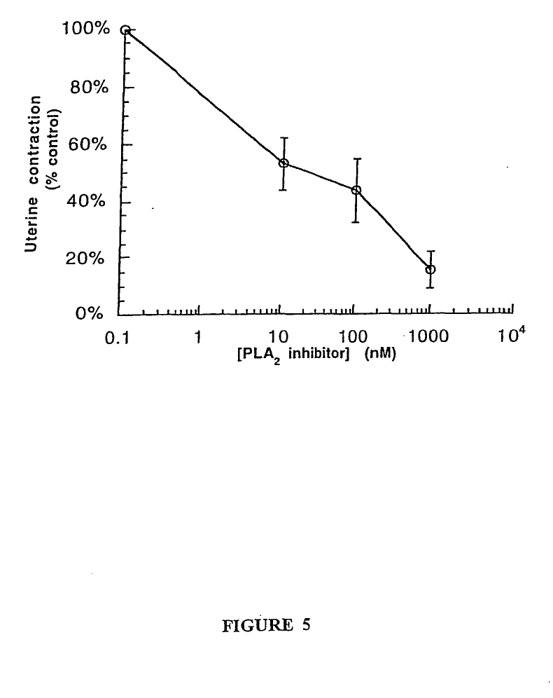

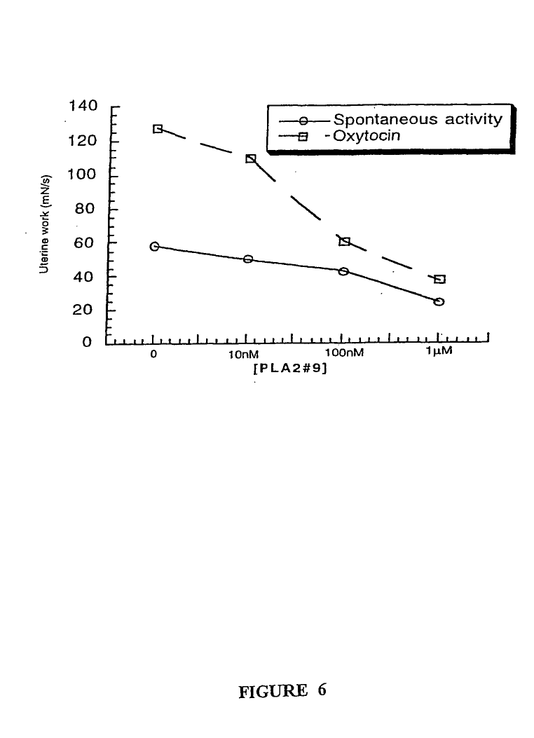

In this rat uterine model we find that non-steroidal antiinflammatory drugs (NSAIDs) such as flunixin, meglumine, piroxicam and naproxen do suppress spontaneous and oxytocin-induced uterine contractions but only at micromolar to millimolar drug concentrations (Table 1). The inventors also found that compound 5, a potent non-peptide inhibitor of human non-pancreatic sPLA₂ (type IIa), is a 1,000 fold more potent inhibitor (EC₅₀ 1-20 nM) of uterine contractions compared to NSAIDs (FIGS. 4 and 5). Compound 9, containing the poorly bioavailable phosphonate group, was also an inhibitor of rat uterine contractions but only at a ten-fold higher concentration than used for 5 (EC₅₀ 80-200 nM) (Table 1). Dose response profiles for inhibition of rat uterine contractions are shown in FIGS. 4 and 6.

The two inhibitors of human sPLA₂ (type IIa) showed greatest activity when examined with uteri treated with oestrogen plus progesterone (average 53% inhibition at 1 nM) while the NSAIDs showed least activity in these uteri (average 30% inhibition at 10 μM). These data suggest that with this hormone treatment prostaglandin is not the dominant mediator of uterine contraction, but other products of the arachidonic acid cascade (possibly LTs or HETEs) or possibly PAF (or combinations thereof) mediate oxytocin-induced contractions. Maximum inhibition of contraction by all drugs was seen in progesterone-primed tissue with PLA₂ inhibitors averaging 75% inhibition at 10 nM and NSAIDs 90% inhibition at 100 nM. Least inhibition was seen in oestrogen-primed uteri where average inhibition was 42% at a concentration of 100 nM of each drug. In these two stages it appears that prostaglandins are significant mediators of contraction, but are not the only mediators. The data from the control rats were variable and it was not possible to draw any conclusions about the likely mediators of contraction in this group.

The in vitro data demonstrated that blocking eicosanoid synthesis could reduce the intensity of uterine work, but the hormone profile had a significant influence on the proportional contribution to uterine contractions of various eicosanoids or other mediators derived from PLA₂. The three NSAIDs tested (flunixin, piroxicam, ketoprofen) also inhibit rat uterine contractions but overall showed much less activity than either of the two sPLA₂ inhibitors 5 and 9. Not wishing to be bound by any one particular theory or mode of action, it is considered that the sPLA₂ inhibitors block the synthesis of mediators including prostaglandins, leukotrienes, platelet activating factor and HETEs, and consequently, inhibit uterine contractions induced by such mediators.

The greatly improved activity for sPLA₂ inhibitors over conventional NSAIDs in the inhibition of uterine contractions suggests a very real prospect of using human sPLA₂ inhibitors as more effective anti-inflammatory agents to treat human dysmenorrhoea. Secretory PLA₂ isozymes (type II, IV) have been reported in gestational tissues and are released by intrauterine tissues (Rice, G. E., 1995, Reprod. Fert. Dev. 7: 1471-1479). The inhibition of this enzyme by drugs that specifically target human sPLA₂ enzyme activity have the potential to give relief of symptoms to a greater number of women who have primary dysmenorrhoea, menstrual migraine and menorrhagia than are currently relieved by conventional treatments such as NSAIDs and hormones. These drugs would also have powerfull antiinflammatory activity making them also suitable for the treatment of inflammatory diseases causing secondary dysmenorrhoea.

Despite clear evidence that prostanoids stimulate uterine contraction, and that NSAIDs can be effective to some degree in some women suffering from dysmenorrhoea, the literature is conflicting as to the mechanism of action of NSAIDs in inhibiting uterine contractions. Several publications claim that NSAIDs inhibit voltage-sensitive Ca channels to exert their tocolytic effect (Sawdy, R. et al, 1998, Br. J. Pharmacol. 125: 1212-1217). Since a significant proportion of women suffering from dysmenorrhoea fail to respond to NSAID therapy, it may be anticipated that other mediators of uterine contraction are involved.

This present invention, therefore, demonstrates that inhibitors of the human sPLA₂ (type IIa) enzyme are superior to NSAIDs, both in terms of potency and in the timing of their use with respect to sex hormone levels, for the inhibition and treatment of uterine contractions and associated disorders arising from the menstrual cycle. These disorders include, but are not limited to, dysmenorrhoea, menstrual migraine and menorrhagia. Human sPLA₂ inhibitors, as described herein, are considered to specifically inhibit the formation of mediators that cause uterine contraction directly or indirectly via the release of other mediators. These mediators, which include PGs, LTs, HETEs and PAF, may alone, or in combination, promote disorders associated with the menstrual cycle, which may be refractory to other currently available treatments, such as NSAIDs or hormones. The present invention, therefore, proposes a novel treatment for disorders arising from the menstrual cycle, be they of an inflammatory nature or alternatively arising from physiological regulators released which produce painful stimuli.

### Example 3

Synthesis of Compound 5

(S)-5-(4-Benzyl-phenylsulfanyl)-4-(7-phenyl-heptanoylamino)-pentanoic acid (5)

The synthesis of (5) is shown in Scheme 1. Commercially available 2-tert-butoxycarbonylamino-pentanedioic acid 5-cyclohexyl ester (10) was chosen as the starting material in the synthesis of (7). In contrast Dennis et al utilised the methyl ester. (Cha, S. S. et al, 1996, J. Med. Chem., 39: 3878-81) However the chemical transformations used in both syntheses are similar.

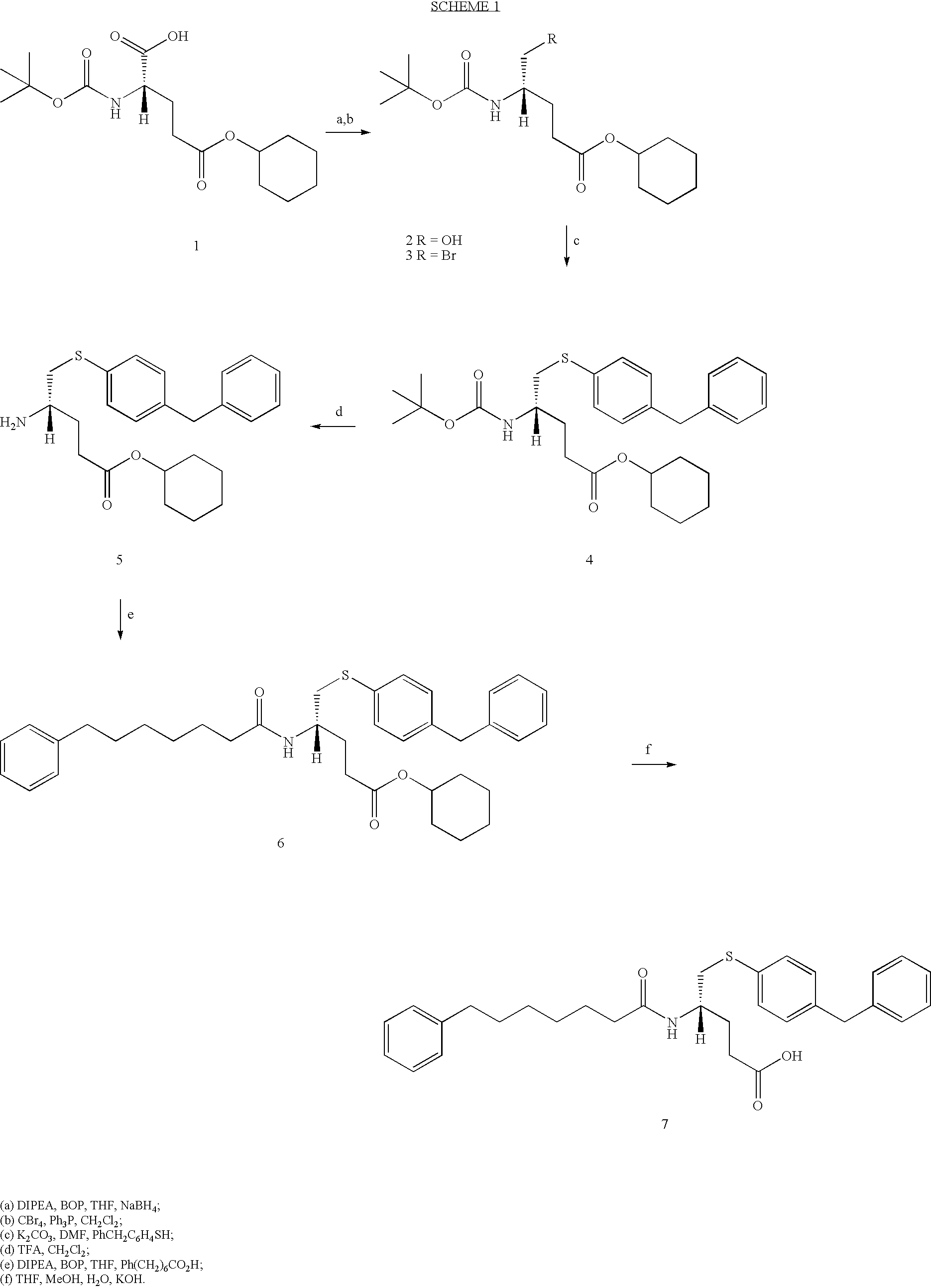

Reduction of (10) to the alcohol (11) was carried out via activation of (10) as the BOP ester and reduction with NaBH₄ in THF.(Ref) The alcohol was transformed into the bromide (12) via standard conditions using Ph₃P and CBr₄ in CH₂Cl₂ in 47% yield. This contrasts with the method used by Dennis et al involving N,N′-dicyclohexylcarbodiimide methiodide which was unsuccessful in our hands.

Compound (12) was coupled with 4-benzyl-benzenethiol with K₂CO₃ in DMF to afford (13). Boc deprotection of (13) with TFA in CH₂Cl₂ afforded the amine (14) quantitatively, which was coupled with BOP-activated 7-phenylheptanoic acid, giving amide (15) in 84% yield. Hydrolysis of (15) with KOH and HPLC purification yielded the required acid (5) in 41% yield.

(S)-4-tert-Butoxycarbonylamino-5-hydroxy-pentanoic acid cyclohexyl ester (11)

DIPEA (1.27 mL, 0.942 g, 7.30 mmol) was added to a mixture of 10 (2 g, 6.08 mmol) and BOP (2.96 g, 6.69 mmol) in THF (25 mL) at room temperature. The mixture became homogenous and was stirred over 10 min. NaBH₄ (231 mg, 6.08 mmol) was added quickly and the mixture stirred overnight. The solvent was removed and the residue taken up into Et₂O. The organic layer was washed successively with 5% aqueous HCl and saturated NaHCO₃. The solution was dried over NA₂SO₄ and the solvent removed. The residue was subject to silica gel chromatography (50% EtOAc/petroleum ether) to yield the title compound as a clear colourless oil (1.07 g, 56%). ¹H NMR (500 MHz, CDCl₃) δ 1.21-1.52 (16H, m), 1.67-1.85 (6H, m), 2.35 (2H, m), 3.51-3.60 (3H, m), 4.72 (1H, m), 4.88 (1H, m). ¹³C NMR (500 MHz, CDCl₃) δ 23.7, 25.3, 26.1, 28.3, 31.2, 31.5, 54.4, 65.1, 73.0, 79.5, 156.2, 173.3.

(S)-5-Bromo-4-tert-butoxycarbonylamino-pentanoic acid cyclohexyl ester (12)

A solution of 11 (1.91 g, 6.08 mmol) and CBr₄ (2.52 g, 7.60 mmol) in CH₂Cl₂ (12 mL) was cooled to 0° C. Ph₃P (2.4 g, 9.15 mmol) was added over 20 min. The solvent was removed and the residue taken up into Et₂O. Upon washing with water and brine the solution was dried and the solvent removed. The residue was subjected to silica gel chromatography (25% EtOAc/petroleum ether, Rf 0.73) yielding the title compound as a colourless oil (1.075 g, 47%). ¹H NMR (CDCl₃) δ 1.2-1.9 (21H, m), 2.34 (2H, m), 3.47 (2H, m), 3.8 (1H, m), 4.72 (1H, m), 4.8 (1H, m). ¹³C NMR (CDCl₃) δ 23.6, 25.2, 28.2, 31.0, 31.5, 38.0, 50.0, 72.9, 79.6, 155.1, 172.3.

(S)-5-(4-Benzyl-phenylsulfanyl)-4-tert-butoxycarbonylamino-pentanoic acid cyclohexyl ester (13)

A solution of 12 (412 mg, 1.09 mmol) 4-benzyl-benzenethiol (327 mg, 1.64 mmol) and K₂CO₃ (226 mg, 1.64 mmol) in DMF (2.5 mL) was stirred at room temperature for 24 h. Et₂O was added, the solution was washed with water and then dried with NA₂SO₄. The solvent was removed and the crude material purified by column chromatography (silica gel, 25% EtOAc/petroleum ether, Rf 0.73) giving a crystalline solid (541 mg, quantitative). ¹H NMR (CDCl₃) δ 1.2-2.1 (21H, m), 2.34 (2H, t), 3.05 (2H, m), 3.8 (1H, m), 3.9 (2H, s), 4.77 (2H, m), 7.1-7.35 (9H, m). ¹³C NMR (CDCl₃) δ 23.6 25.2, 28.2, 28.6, 31.2, 31.4, 39.4, 41.2, 50.0, 72.5, 78.9, 125.9, 128.3, 128.7, 129.4, 129.8, 133.4, 139.2, 140.6, 155.2, 172.5.

(S)-4-Amino-5-(4-benzyl-phenylsulfanyl)-pentanoic acid cyclohexyl ester (14)

TFA (9 mL) was added to a solution of 13 (541 mg, 1.09 mmol) in CH₂Cl₂ (25 mL). The solution was stirred for 30 min and the CH₂Cl₂/TFA removed under vacuo. The residue was subject to silica gel chromatography (20% MeOH/EtOAc, Rf 0.49) to provide the title compound (432 mg, quantitative). ¹H NMR (CDCl₃) δ 1.2-1.8 (10H, m), 2.04 (2H, m), 2.43 (2H, m), 3.3 (1H, m), 3.14 (2H, m), 3.91 (2H, s), 4.7 (1H, m), 7.1-7.34 (9H, m), 8.15 (2H, br s). ¹³C NMR (CDCl₃) δ 23.4, 25.1, 26.7, 30.4, 31.2, 36.9, 41.3, 50.8, 73.7, 126.1, 128.4, 128.7, 129.8, 130.3, 131.1, 140.3, 140.8, 172.7.

(S)-5-(4-Benzyl-phenylsulfanyl)-4-(7-phenyl-heptanoylamino)-pentanoic acid cyclohexyl ester (15)

A solution of 7-phenylheptanoic acid (230 mg, 1.117 mmol), BOP (543 mg, 1.23 mmol) and DIPEA (233 μL, 173 mg, 1.34 mmol) in THF (10 mL) was stirred for 10 min at room temperature. To this a solution of 14 (432 mg, 1.09 mmol) and DIPEA (1 mL, 5.75 mmol) in THF (5 mL) was added. The mixture was stirred overnight. The solvent was removed and the residue taken up in EtOAc. The organic layer was washed successively with aqueous 1M HCl, saturated NaHCO₃ and brine. The solution was dried over NA₂SO₄ and the solvent removed. The residue was subject to silica gel chromatography (30% EtOAc/petroleum ether) to yielded the title compound (534 mg, 84%). ¹H NMR (CDCl₃) δ 1.1-2 (20H, m), 2.07 (2H, t), 2.6 (2H, t), 2.95 (2H, m), 3.9 (2H, s), 4.8 (1H, m), 6.3 (1H, m), 7.1-7.4 (14H, m). ¹³C NMR (CDCl₃) δ 23.3, 24.9, 25.2, 27.9, 28.6, 28.8, 30.9, 31.2, 35.5, 36.2, 38.4, 40.9, 48.4, 72.4, 125.2, 125.7, 127.8, 127.9, 128.1, 128.5, 129.2, 133.2, 138.9, 140.3,142.2, 172.4, 172.7.

(S)-5-(4-Benzyl-phenylsulfanyl)-4-(7-phenyl-heptanoylamino)-pentanoic acid (5)

A solution of 15 (534 mg, 0.913 mmol) and KOH (64 mg, 85%, 0.959 mmol) in aqueous THF (15 mL) was stirred overnight. The residue was subject to HPLC (30% aqueous CH₃CN) to provide the title compound, upon lyophilisation, as a white powder (206 mg, 41%), mp 89-91.5° C. (lit. 85-88° C.)³. ¹H NMR (500 MHz, CDCl₃) δ 1.29 (4H, m), 1.5-1.62 (4H, m), 1.80 (1H, m), 1.94 (1H, m), 2.04 (2H, t, J=7.6 Hz), 2.36 (2H, t, J=6.55 Hz), 2.58 (2H, t, J=7.7 Hz), 3.06 (2H, m), 3.92 (2H, s), 4.19 (1H, m), 5.67 (1H, D, J=8.55 Hz), 7.09-7.31 (14H, m). ¹³C NMR (500 MHz, CDCl₃) δ 25.4, 28.6, 28.9, 39.0, 30.8, 31.2, 35.8, 36.6, 39.2, 41.4, 48.8, 125.6, 126.2, 128.2, 128.4, 128.5, 128.9, 129.7, 130.1, 133.0, 139.9, 140.6,142.6, 173.7,176.8. ESMS 504 (M+H)⁺.

### Example 4

Synthesis of Compound 9

[3-(1-Benzyl-3-carbamoylmethyl-2-methyl-1H-indol-5-yloxy)-propyl]-phosphonic acid] (9).

The synthesis of (9) is shown in Scheme 2. A standard Fischer indole synthesis in ethanolic HCl yielded the 2,3,5-substituted indole (16) in 31% yield. Benzylation of (16) gave (17) which was demethylated with BBr₃ to produce (18). Alkylation gave (19), which was hydrolysed to (20). Activation of the acid (20) with HBTU and treatment with NH₄OH yielded the phosphonic ester (21), which in turn was converted to the acid (9) with Me₃SiI in CH₂Cl₂.

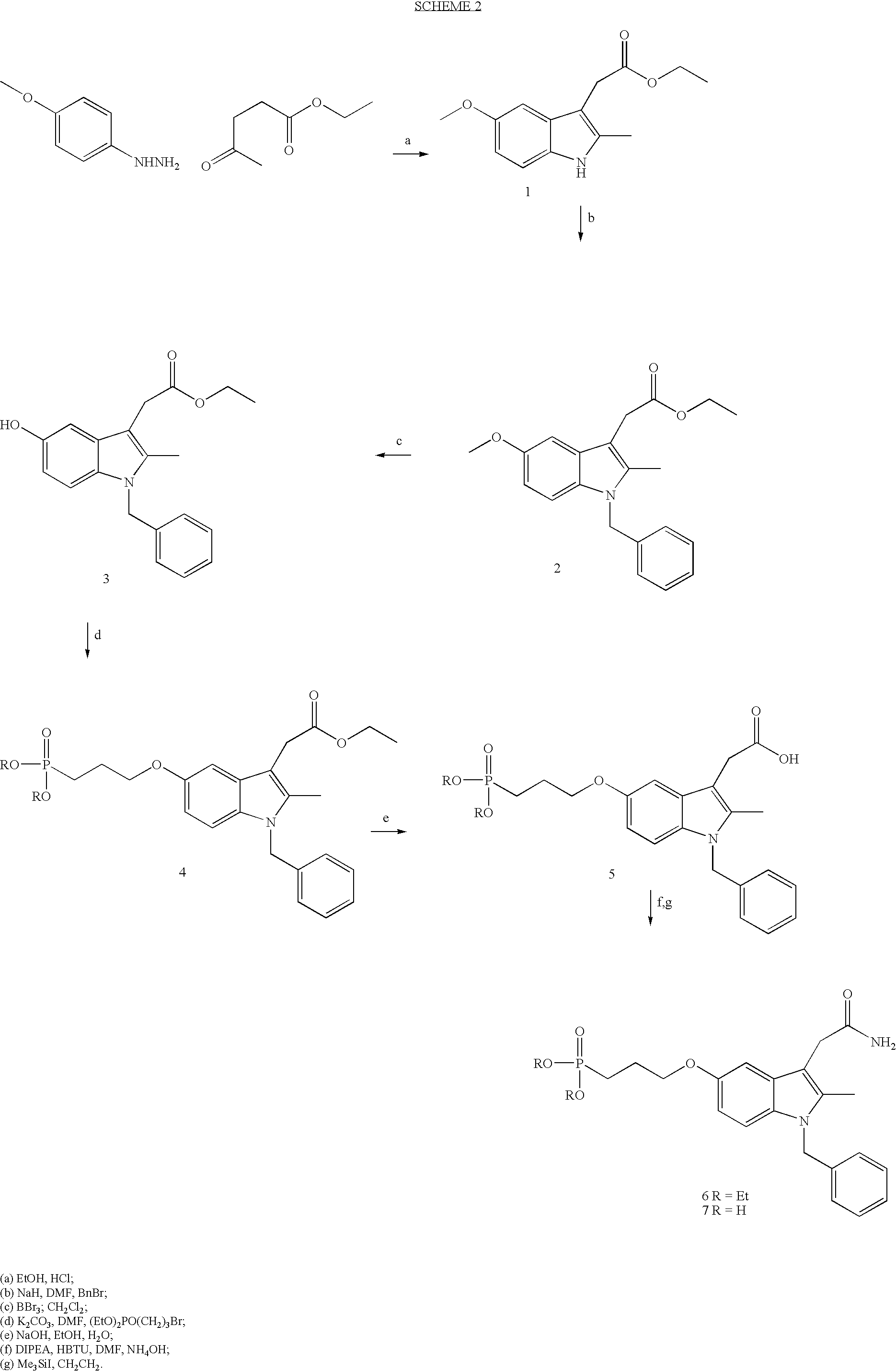

(5-Methoxy-2-methyl-1H-indol-3-yl)-acetic acid ethyl ester (16)

4-Methoxyphenylhydrazine hydrochloride (7 g, 40.1 mmol) and ethyl levulinate (5.73 g, 40.1 mmol) were refluxed in ethanolic HCl (2 M, 180 mL) for 3 h. The solvent was removed, diluted with water and extracted with Et₂O. The Et₂O was washed with 20% aqueous NaHCO₃, dried and filtered and the solvent removed. The crude material was subject to silica gel chromatography (20% Et₂O/petroleum ether) to afford the title compound (3.03 g, 31%). ¹H NMR (CDCl₃) δ 1.24 (3H, t, J=7.1 Hz, CO₂CH₂CH₃), 2.36 (3H, s, CH₃), 3.64 (2H, s, CH₂CO₂CH₂CH₃), 3.85 (3H, s, OCH₃), 4.12 (2H, q, J=7.1 Hz, CO₂CH₂CH₃), 6.76 (1H, dd, J=2.4, 8.7 Hz), 7.00 (1H, d, J=2.4 Hz), 7.13 (1H, d, J=8.7 Hz).

(1-Benzyl-5-hydroxy-2-methyl-1H-indol-3-yl)-acetic acid ethyl ester (18)

To 16 (1.245 g, 5.04 mmol) in dry DMF (20 mL) at 0° C. under nitrogen was added NaH (60%, 0.2 g, 5.04 mmol). The mixture was stirred to room temperature over 30 min. BnBr (0.86 g, 0.60 mL, 5.04 mmol) was added dropwise by syringe. The reaction was complete in 3 h and quenched with water. The mixture was extracted with EtOAc that was washed with brine and then dried with NA₂SO₄. Filtration and removal of the solvent afforded crude product, which was subjected to column chromatography (15% EtOAc/petroleum ether). Thus 17 was obtained (0.69 g, 41%). To a solution of 17 (670 mg, 1.99 mmol) in CH₂Cl₂ (15 mL) under argon at 0° C. was added dropwise BBr₃ (0.75 g, 0.28 mL, 2.98 mmol). Warming to room temperature the reaction was complete in 2 h. It was cooled on ice and quenched with water. The CH₂Cl₂ was washed with water then brine, dried over NA₂SO₄, filtered and the solvent removed. The crude product was subjected to column chromatography (silica gel, 30% EtOAc/petroleum ether) to afford the title compound (0.31 g, 49%), mp 122-125° C. ¹³C NMR (CDCl₃) δ 10.4, 14.2, 30.9, 46.7, 60.7, 103.0, 103.9, 109.7, 110.5, 125.9, 127.2, 128.5, 128.7, 131.7, 135.3, 137.9, 149.6, 172.2.

{1-Benzyl-5-[3-(diethoxy-phosphoryl)-propoxy]-2-methyl-1H-indol-3-yl}-acetic acid ethyl ester (19)

Compound 18 (0.3 g, 0.93 mmol), K₂CO₃ (0.77 g, 5.59 mmol) and (3-Bromo-propyl)-phosphonic acid diethyl ester (0.24 g, 0.93 mmol) in DMF (5 mL) was stirred at room temperature for 72 h. The reaction mixture was diluted with water and extracted with EtOAc. The EtOAc was washed with water, dried and filtered. Removal of the solvent afforded material which was purified by column chromatography (silica gel, 30% EtOAc/petroleum ether) to give the title compound (0.35 g, 75%). ¹³C NMR (CDCl₃) δ 10.5, 14.2, 16.4 (5.6 Hz), 22.5 (143 Hz), 22.9 (4.4 Hz), 30.8, 46.7, 60.6, 61.5 (6.8 Hz), 68.2 (17 Hz), 101.7, 104.3, 109.7, 111.2, 125.9, 127.2, 128.2, 128.7, 131.7, 135.0, 137.9, 153.2, 171.9.

[3-(1-Benzyl-3-carbamoylmethyl-2-methyl-1H-indol-5-yloxy)-propyl]-phosphonic acid diethyl ester (21)

A solution of 19 (200 mg, 0.39 mmol) and NaOH (17 mg, 0.41 mmol) in EtOH/water (4:1, 10 mL) was heated under reflux for 45 min. The solvent was removed, water added and the solution washed with EtOAc. The aqueous layer was acidified with HCl (1M) and extracted with EtOAc. The EtOAc was washed with water, brine and dried with NA₂SO₄. Filtration and removal of the solvent yielded 20, mp 106-111° C. To a solution of 20 (80 mg, 0.17 mmol) in DMF was added HBTU (0.5 M in DMF, 340 μL, 0.17 mmol) and DIPEA (22 mg, 30 μL, 0.17 mmol). After 10 min NH₄OH (14.7 M, 23 μL, 0.33 mmol) was added and the solution stirred for 3 h. The reaction mixture was diluted with EtOAc, washed successively with NaHCO₃, water and saturated NaCl. Drying with NA₂SO₄ and removal of the solvent afforded the title compound (66 mg, 82%), mp 130-133° C. ¹³C NMR (CDCl₃) δ 10.3, 16.4, 16.5, 22.3 (142.1 Hz), 22.8, 32.2, 46.8, 61.5, 61.6, 68.2 (16.6 Hz), 101.2, 104.9, 110.1, 111.8, 125.9, 127.4, 127.7, 128.8, 131.9, 135.3, 137.5, 153.5, 174.3.

[3-(1-Benzyl-3-carbamoylmethyl-2-methyl-1H-indol-5-yloxy)-propyl]-phosphonic acid (9)

Trimethylsilyliodide (49 mg, 35 μL, 0.24 mmol) was added to a solution of 21 (23 mg, 0.049 mmol) in CH₂Cl₂ (1 mL) under nitrogen. The reaction mixture was stirred for 14 h after which time MeOH/water (5 mL, 4:1) was added and the mixture was stirred for an additional 30 min. The solvents were removed and the crude material subject to reverse phase HPLC (CH₃CN/water) to afford the title compound (12 mg, 59%), mp 199-203° C. ¹³C NMR (CD₃OD/DMSO-d₆) δ 10.4, 23.3 (4.6 Hz), 24.3 (141.3 Hz), 31.7, 46.0, 68.3 (16.9 Hz), 102.2, 105.8, 109.9, 110.4, 126.3, 127.1, 128.3, 128.7, 131.4, 135.1, 138.8, 152.7, 173.3.

### Example 5

Assay for Inhibition of Human Non-pancreatic sPLA₂ (Type IIa)

A mixed micelle colorimetric assay utilising a microtitre plate reader was utilised as described by Reynolds, L. J.; Hughes, L. L.; Dennis, E. A. “Analysis of human synovial fluid phospholipase A₂ on short chain phosphatidylcholine-mixed micelles: Development of a spectrophotometric assay suitable for a microplate reader”, Anal. Biochem. 1992, 204, 190-197.

Reagents were obtained commercially in the sPhospholipase A₂ Assay Kit, Cayman Chemical Company MI USA. Buffer (25 mM Tris HCl at pH 7.5, 1 mg/mL BSA, 0.3 mM Triton X-100, 100 mM KCl, 10 mM CaCl₂), substrate (diheptanoyl thio-phosphatidyl choline), DTNB (5,5′-dithiobis(2-nitrobenzoic acid)) and a 96 well plate were all provided. Recombinant hIIa-PLA₂ was provided by the Garvan Institute for Medical Research. The enzyme was found to be homogenous by LCMS and an MS reconstruct yielded a molecular weight of 13,905 g/mole as expected. Standard inhibitor solutions were prepared from anhydrous DMSO. All samples were run in triplicate including the blank and control samples. Data was collected on a Molecular Devices Spectramax™ 250 Microplate Spectrophotometer using Softmax™ Pro Microplate Analysis Software v2.21. The mechanistic details of the assay are shown in Scheme 3.

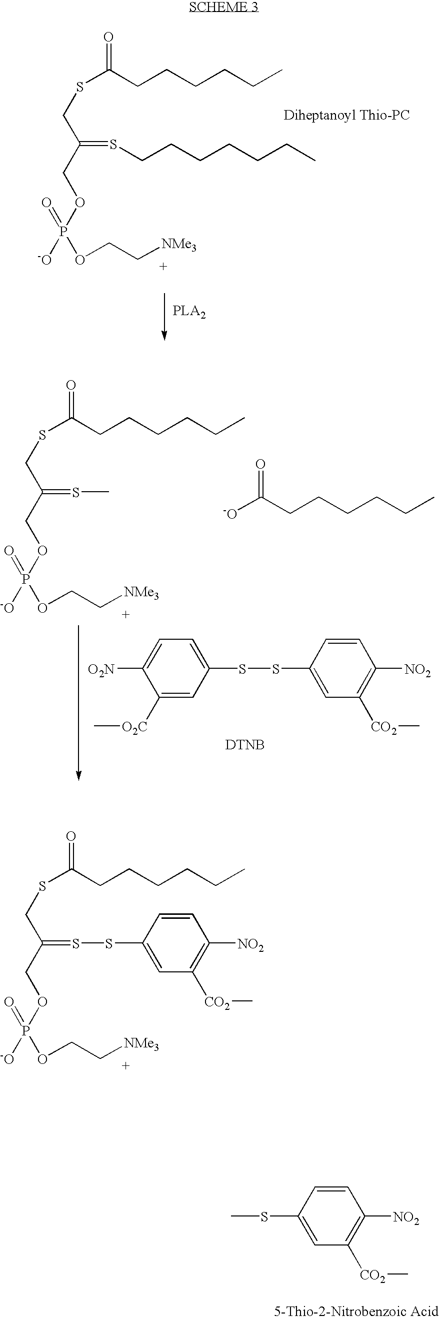

Diphenylheptanoyl Thio-PC is processed by s-PLA₂ and the free thiol produced is detected with DTNB (Ellman's Reagent). 5-Thio-2-nitrobenzoic acid is detected spectrophotometrically at 414 nM. IC50's were determined by assaying inhibitors at a range of concentrations. Inhibitor concentration was plotted as a function of the inverse of the initial velocity (Dixon plot) and extrapolation to the X axis yielded the IC50.

### Example 6

Assays for Dysmenorrhoea

Inhibition of Uterine Contractions in Naturally-cycling Rats

Uteri were removed from rats, divided into 4 segments and placed in organ baths to measure force and frequency of contraction. Spontaneous contractions were measured, then each segment of uterus was challenged with oxytocin (1 μI.U. to 100 mI.U.). The force and frequency of contraction was measured after oxytocin addition and a concentration response curve generated. The drugs—either an sPLA₂ inhibitor or a NSAID—was then added to the bath and allowed to equilibrate for 30 minutes. The drugs were tested at concentrations ranging from 1 nM to 10 μM with one piece of tissue per concentration. The tissue was then challenged with increasing concentrations of oxytocin to generate a second concentration-response curve. The EC₅₀ concentrations in control and drug-treated tissue were determined from the curves and compared.

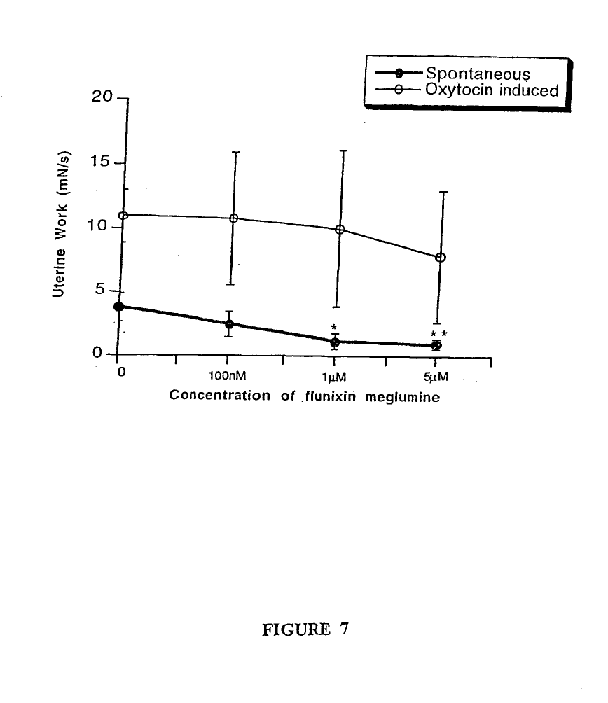

PLA₂ inhibitors 5 and 9 were tested in rat uterus in organ baths to ascertain the potency of these compounds in this preparation. The NSAIDs flunixin meglumine and piroxicam were also tested in the model. The results shown in FIGS. 4-7 indicate that the sPLA₂ inhibitors 5 and 9 are significantly more potent as inhibitors of spontaneous or oxytocin-induced contractions of rat uteri than the NSAIDs flunixin meglumine and piroxicam. The results are summarised in Table 1.

TABLE 1
Summary of activity of PLA₂ inhibitors and NSAIDs
in modifying spontaneous activity and oxytocin-induced
contractions in a rat uterus model of dysmenorrhoea

| | ED₅₀ | ED₅₀ |
|---|---|---|
| | Spontaneous | Oxytocin-induced |
| Drug Treatment | contractions | contractions |
| PLA₂ #5 | 30 nM | 20 nM |
| PLA₂ #9 | 80 nM | 200 nM |
| Flunixin | 1 μM | 1 μM |
| Piroxicam | No effect | No Effect |

Effects of Sex Hormones on the Force of Uterine Contraction

In a separate series of experiments, rats were ovarectomised then treated with sex hormones to ensure animals are at the same stage of the oestrus cycle. Uteri were removed from the rats 4 days after hormone treatment, allowed to equilibrate in the organ baths for 30 minutes, then the intensity of uterine contractions and frequency of the spontaneous contractions was measured. In the oestrogen-primed uteri, spontaneous uterine work was 7.2±1.4 mNs^{31 1}, in progesterone-primed rats, 10±0.9 mNs⁻¹, in oestrogen plus progesterone-primed uteri, 9±1.5 mNs⁻¹ and in control uteri 7.9±0.6 mNs⁻¹. Hormone treatments did not significantly alter the intensity of spontaneous uterine work (ANOVA, n=4-5, P>0.05).

Challenging with oxytocin caused an increase in uterine work over baseline spontaneous activity. The maximum work achieved was with 100 mU oxytocin which resulted in a response of 18 mNs⁻¹±1.1 mNs⁻¹ in the oestrogen plus progesterone treated group (n=5, P<0.001), 15.7 mNs⁻¹±2 mNs⁻¹ in the oestrogen group (n=4, P<0.001), 0.9 mNs⁻¹ (n=5, P>0.05) in the progesterone group and 7.8 mNs⁻¹±0.7 mNs⁻¹ (n=4, P>0.05) in the control group (ANOVA with Tukey post test). These data demonstrate that oestrogen primes the uterus for contraction.

### Example 7

Effects of Anti-inflammatory Drugs on Uterine Activity

sPLA₂ inhibitor 5 and ketoprofen were tested in the model to determine their ability to moderate both spontaneous contractions and contractile activity stimulated by oxytocin.

(i) Control Group

In uteri removed from rats treated with the oil vehicle, 1 nM sPLA₂ inhibitor 5 inhibited spontaneous activity (ANOVA, n=4, P<0.05). When uteri were challenged with oxytocin in the presence of inhibitor 5 (1 nM-1 μM), the drug did not cause a shift in the oxytocin concentration-response curve as seen by changes in the oxytocin EC₅₀ values (ANOVA, n=4, P>0.05). It did, however, cause a decrease in the intensity of the oxytocin response with uterine work being maximally inhibited by 40%±11% (ANOVA, n=4, P<0.01) at 100 nM (Table 2).

TABLE 2
The maximum inhibition of uterine work by a PLA₂
inhibitor and a nonsteroidal antiinflammatory drug in
rat uterus challenged with oxytocin

| | Vehicle | Oestrogen (O) | Progesterone (P) | O + P |
|---|---|---|---|---|
| Ketoprofen | 79% ± 5% | 29% ± 5% | 84% ± 4% | No effect |
| | 100 nM | 100 nM | 100 nM | |
| | n = 4, P < 0.001 | n = 4, P < 0.01 | n = 5, P < 0.01 | n = 5, P > 0.05 |
| sPLA₂ | 40% ± 11% | 34% ± 5% | 65% ± 6% | 62% ± 4% |
| Inhibitor 5 | 100 nM | 100 nM | 10 nM | 1 nM |
| | n = 4, P < 0.01 | n = 4, P < 0.005 | n = 5, P < 0.01 | n = 5, P < 0.0001 |

The values are the mean percentages and SEMs of inhibition of uterine work in drug-treated uteri compared to the same uteri before drug. Also shown is the concentration at which the drug had this maximum inhibitory effect.
N = number of experiments.
P = statistical significance of data.

(ii) Progesterone Treatment

In progesterone treated uteri, inhibitor 5 (1 nM-1 μM) did not significantly inhibit spontaneous activity, nor did it shift the oxytocin concentration-response curves, with no detectable change in the EC₅₀ values (ANOVA, n=5, P>0.05). It did cause a decrease in the intensity of the oxytocin response with the maximum inhibition of uterine work of 65%±6% (ANOVA, n=5, P<0.01) at 10 nM (Table 2). Ketoprofen (100 nM) inhibited 84% of the uterine contractions with this hormone treatment.

(iii) Oestrogen Treatment

The spontaneous activity of uteri from rats with this hormone treatment was inhibited by 10 nM PLA₂ inhibitor 5 (ANOVA, n=4, P<0.05). Inhibitor 5 (1 nM-1 μM) did not alter the oxytocin EC₅₀ values in oestrogen treated uteri (ANOVA, n=4, P>0.05). There was a maximum decrease in the intensity of the oxytocin response of 34%±5% (ANOVA, n=4, P<0.005) at 100 mM (Table 2). Ketoprofen (100 nM) inhibited only 29% uterine contractions with this hormone treatment.

(iv) Oestrogen and Progesterone Treatment

Inhibitor 5 (1 nM-1 μM) did not inhibit spontaneous activity, or oxytocin EC₅₀ values in uteri treated with both oestrogen and progesterone (ANOVA, n=5, P>0.05). The drug reduced the maximum oxytocin response by 62%±4% (ANOVA, n=5, P<0.0001) at a concentration of 1 nM (Table 2). Ketoprofen (up to 10 μM) had no inhibitory effect for this hormone treatment.

In the presence of progesterone plus oestrogen, which more closely reproduces the natural sexual cycle than either oestrogen or progesterone alone, the sPLA₂ inhibitor 5 at 1 nM concentration was a potent inhibitor of rat uterine contractions.

The disclosure of every publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### Bibliography

Balboa, M. A., Balsinde, J., Winstead, M. V., Tischfield, J. A. and Dennis, E. A. 1996. Novel group V phospholipase A₂ involved in arachidonic acid mobilisation in murine P388D₁ macrophages. J. Biol. Chem. 271 (50): 32381-32384.

Balsinde J, Balboa M A, Insel P A, Dennis E A., 1999. Regulation and inhibition of phospholipase A₂. Annu Rev Pharmacol Toxicol.; 39:175-89.

Benedetto, C., 1989. Eicosanoids in primary dysmenorrhoea, endometriosis and menstrual migraine. Gynecol Endocrinol, 3: 71-94.

Bieglmayer, C., Hofer, G., Kainz, C., Reinthaller, A., Kopp, B. and Janisch, H., 1995. Concentrations of various arachidonic acid metabolites in menstrual fluid are associated with menstrual pain and are influenced by hormonal contraceptives. Gynecol Endocrinol, 9: 307-312.

Bonner, J. and Sheppard, B. L., 1996. Treatment of menorrhagia during menstruation: randomised controlled trial of ethamsylate, mefenamic acid and tranexamic acid. BMJ, 313: 579-582.

Bryant, M. D.; Flick, K. E.; Koduri, R. S.; Wilton, D. C.; Stoddard, B. L.; Gelb, M. H. 1999. Bioorg Med Chem Lett, 9: 1097-102.

Cha, S. S.; Lee, D.; Adams, J.; Kurdyla, J. T.; Jones, C. S.; Marshall, L. A.; Bolognese, B.; Abdel-Meguid, S. S.; Oh,. B. H., 1996. High-resolution X-ray crystallography reveals precise binding interactions between human nonpancreatic secreted phospholipase A₂ and a highly potent inhibitor (FPL67047XX). J. Med. Chem., 39:, 3878-81.

Chen, Y.; Dennis, E. A. 1998. Expression and characterisation of human group V phospholipase A₂. Biochim. Biophys Acta, 1394: 57-64.

Dawood, M. Y. 1988. Nonsteroidal anti-inflammatory drugs and changing attitudes towards dysmenorrhoea. Am J Med, 84: 23-29

Hanasaki, K., Ono, T., Saiga, A., Morioka, Y., Ikeda, M., Kawamoto, K., Higaslino, K., Nakano, K., Yamada, K., Ishizaki, J., and Arita, H. 1999. Purified group X secretory phospholipase A₂ induced prominent release of arachidonic acid from human myeloid leukemia cells. J. Biol. Chem., 274 (48): 34203-34211.

Kitadokoro, K., et al. 1998. Crystal structure of human secretory phospholipase A₂-IRA complex with the potent indolizine inhibitor 120-1032. J. Biochem. 123: 619-623.

Lopez Bernal A., Canete Soler R and Turnbull, A. C. 1989. Are leukotrienes involved in human uterine contractility? Br J Obstet Gynaecol, 96: 568-73

Murakami, M., Kambe, T., Shimbara, S., Higashino, K., Hanasaki, K., Arita, H., Horiguchi, M., Arita, M., Arai, H., Inoue, K. and Kudo, I. 1999. Different functional aspects of the group II subfamily (types Iia and V) and type X phospholipase A₂s in regulating arachidonic acid release and prostaglandin generation. J. Biol. Chem. 274 (44): 31435-31444.

Nattero, G., Allais, G., De Lorenzo, C., Benedetto, C., Zonca, M., Melzi, E. and Massobrio. 1989. Relevence of prostaglandins in true menstrual migraine. Headache 29: 233-238

Nigam, S., Benedetto, C., Zonca, M., Leo-Rossberg, I., Lubbert, H. and Hammerstein, J. 1991. Increased concentrations of eicosanoids and platelet-activating factor in menstrual blood from women with primary dysmenorrhoea. Eicosanoids, 4: 137-141,

Oh, B.-H. 1995. Acta Cryst, D51: 140-144.

Pisabarro, M. T., Ortiz, A. R., Palomer, A., Cabre, F., Garcia, L., Wade, R. C., Gago, F., Mauleon, D., Carganico, G. 1994. J. Med. Chem. 37: 337-41.

Prigent, A., Fayard, J. M., Pageaux, J. F., Lagarde, M., Laugier, C and Cohen, H. 1994. Prostaglandin E2 production by uterine stromal cell line UIII: regulation by estradiol and evidence of an ethanol action. Prostaglandins, 47: 451-66

Reynolds, L. J.; Hughes, L. L.; Dennis, E. A. 1992. Analysis of human synovial fluid phospholipase A₂ on short chain phosphatidylcholine-mixed micelles:Development of a spectrophotometric assay suitable for a microplate reader. Anal. Biochem. 204: 190-197.

Rice, G. E. 1995. Secretory type II PLA₂ and the generation of intrauterine signals. Reprod. Fertil. Dev. 7: 1471-9.

Scott, D. L., White, S. P., Browning, J. L., Rosa, J. J., Gelb, M. H., Sigler, P. B. 1991. Structures of Free and Inhibited Human Secretory Phospholipase A₂ from inflammatory exudate. Science 254: 1007-10.

Schevitz, R. W., Bach, N. J., Carlson, D. G., Chirgadze, N. Y., Clawson, D. K., Dillard, R. D., Draheim, S. E., Hartley, L. W., Jones, N. D., Mihelich, E. D., Olkowski, J. L., Synder, D. W., Sommers, C. and Wery, J.-P. 1995. Structure-based design of the first potent and selective inhibitor of human non-pancreatic secretory phospholipase A₂. Nat. Struct. Biol. 2 (6): 458-65.

Thunnissen, M. M., Ab, E., Kalk, K. H., Drenth, J., Dijkstra, B. W., Kuipers, O. P., Dijkman, R., de Haas, G. H., Verheij, H. M. 1990. Nature 347: 689-91.

van Eijkeren, M. A., Christiaens, G. C., Scholten, P. C. and Sixma, J. J. 1992. Menorrhagia. Current drug treatment concepts. Drugs 43: 201-209

Whittle, B. J. 1979. Prostaglandin synthetase inhibitors. Drugs which affect arachidonic acid metabolism. Acta Ostet Gynecol Scayid Suppl 87: 21-26.

Yu, L., Dennis, E. A. 1992. J. Am. Chem. Soc. 114: 8757-8763.

Zahradnik, H. P. & Brechwood, M. 1984. Contribution to the pathogenesis of dysmenorrhoea. Arch Gynecol 236: 99-108.

1. A method for the treatment and/or prophylaxis of a condition selected from the group consisting of dysmenorrhoea, menstrual migraine and menorrhagia, said method comprising administering to a patient in need of such treatment or prophylaxis an effective amount of a composition comprising a phospholipase inhibitor, together with a pharmaceutically acceptable carrier.

2. The method of claim 1, wherein said inhibitor is a phospholipase A₂ inhibitor.

3. The method of claim 1, wherein said inhibitor is a secretory phospholipase A₂ inhibitor (sPLA₂).

4. The method of claim 1, wherein said inhibitor is a non-pancreatic sPLA₂ inhibitor.

5. The method of claim 4, wherein said inhibitor is a human non-pancreatic sPLA₂ inhibitor.

6. The method of claim 4, wherein said inhibitor is a type IIa non-pancreatic sPLA₂ inhibitor.

7. The method of claim 1, wherein the composition is administered prior to the onset of menstruation.

8. The method of claim 1, wherein the composition is administered topically, locally or transdermally.

9. The method of claim 8, wherein the composition is administered in association with an intravaginal or intracervical device.

10. The method of claim 9, wherein the device is selected from a tampon, diaphragm, sponge, or membrane.

11. The method of claim 8, wherein the composition is administered topically to the surface of the lower abdomen.

12. The method of claim 1, wherein the composition is administered orally.

13. Use of a composition comprising a phospholipase inhibitor in the preparation of a medicament for treating a condition selected from the group consisting of dysmenorrhoea, menstrual migraine and menorrhagia.

14. The use of claim 13, wherein said inhibitor is a phospholipase A₂ inhibitor.

15. The use of claim 13, wherein said inhibitor is a secretory phospholipase A₂ inhibitor (sPLA₂).

16. The use of claim 13, wherein said inhibitor is a non-pancreatic sPLA₂ inhibitor.

17. The use of claim 13, wherein said inhibitor is a human non-pancreatic sPLA₂ inhibitor.

18. The use of claim 13, wherein said inhibitor is a type IIa non-pancreatic sPLA₂ inhibitor.

19. The use of claim 13, wherein the medicament is formulated for topical, local or transdermal administration.

20. The use of claim 19, wherein the medicament is formulated for application in association with an intravaginal or intracervical device.

21. The use of claim 20, wherein the device is selected from a tampon, diaphragm, sponge, or membrane.

22. The use of claim 19, wherein the medicament is formulated for topical application to the surface of the lower abdomen.

23. The use of claim 13, wherein the medicament is formulated for oral administration.
